(19) United States
(12) Patent Application Publication
(10) Pub. No.: US 2008/0214943 A1
(43) Pub. Date: Sep. 4, 2008

(54) Detection of Blood Flow Using Emitted Light Absorption

(75) Inventors: Tomas Kara, Rochester, MN (US); Jiri Nykodym, Rochester, MN (US); Charles J. Bruce, Rochester, MN (US); Paul Friedman, Rochester, MN (US); Kalpaki L. Venkatachalam, Rochester, MN (US); Virend K. Somers, Rochester, MN (US)

Correspondence Address: QUARLES & BRADY LLP, 411 E. WISCONSIN AVENUE, SUITE 2040, MILWAUKEE, WI, 53202-4497, US

(73) Assignee: MAYO FOUNDATION FOR MEDICAL EDUCATION AND RESEARCH, Rochester, MN (US)

(21) Appl. No.: 11/720,566

(22) PCT Filed: Nov. 17, 2005
(86) PCT No.: PCT/US05/42017

§ 371 Date: May 31, 2007

Related U.S. Application Data
(60) Provisional application No. 60/632,388, filed on Dec. 2, 2004.

(51) Int. Cl. A61B 5/026

(52) U.S. Cl. 600/504

(57) ABSTRACT

Perfused tissue is illuminated and light passing through the tissue or reflected from it is detected to produce an electrical signal. Amplitude pulses corresponding to the subject's heart beat are detected in the electrical signal and the areas of these pulses are calculated to produce blood flow values indicative of the blood volume pumped by the heart. The blood flow values may be used alone or in combination with other measured cardiac parameters to evaluate cardiac function.

### CROSS-REFERENCE TO RELATED APPLICATIONS

This application is based on U.S. Provisional Patent Application Ser. No. 60/632,388 filed on Dec. 2, 2004 and entitled “DETECTION OF BLOOD FLOW USING EMITTED LIGHT ABSORPTION”.

### BACKGROUND OF THE INVENTION

The field of the invention is optical measuring techniques for determining desired parameters of a subject's blood using non-invasive or semi-invasive methods.

Optical methods of determining the chemical composition of blood are typically based on spectrophotometric measurements enabling the indication of the presence of various blood constituents based on known spectral behaviors of these constituents. These spectrophotometric measurements may be performed in a non-invasive manner or in a semi-invasive manner.

There are a number of medical applications in which blood parameters are measured. These include: cardiac monitoring systems used in hospitals; portable cardiac monitor and recorder systems commonly referred to as “Holters”; pacemakers; and cardioverters/defibrillators.

The non-invasive optical measurements may be divided into two main groups based on different methodological concepts. The first group represents a so-called “DC measurement technique”, and the second group is called “AC measurement technique”. According to the DC measurement technique, any desired location of a blood perfused tissue is illuminated by the light of a predetermined spectral range, and the tissue reflection and/or transmission effect is studied. Although this technique provides a relatively high signal-to-noise ratio, as compared to the AC measurement technique, the results of such measurements depend on all the spectrally active components of the tissue (i.e., skin, blood, muscles, fat, etc.), and therefore need to be further processed to separate the “blood signals” from the detected signals.

The AC measurement technique focuses on measuring only the “blood signal” of a blood perfused tissue illuminated by a predetermined range of wavelengths. To this end, what is actually measured is a time dependent component only of the total light reflection or light transmission signal obtained from the tissue. A typical example of the AC measurement technique is the known method of pulse oximetry, wherein a pulsatile component of the optical signal obtained from a blood perfused tissue is utilized for determining arterial blood oxygen saturation. In other words, the difference in light absorption of the tissue measured during the systole and the diastole is considered to be caused by blood that is pumped into the tissue during the systole phase from arterial vessels, and therefore has the same oxygen saturation as in the central arterial vessels.

The measurement of blood parameters in conjunction with ECG monitoring and analysis is well known. The measurement of oxygen saturation in connection with ECG monitoring is disclosed in U.S. Pat. Nos. 4,967,748 and 5,176,137. The oxygen saturation information is used along with the ECG information to signal a compromising ventricular tachycardia or fibrillation event. In some applications such as a Holter or bedside monitor, the illumination device and detector may be deployed in a non-invasive manner (e.g., on a finger or ear lobe), whereas in other applications, such as an implantable cardioverter/defibrillator or pacemaker, these devices may be deployed invasively. For example, U.S. Pat. No. 5,601,611 discloses the deployment of an illumination device and detector in the patient's heart to gather blood flow information used to determine the nature of an arrhythmia detected by ECG signals.

### SUMMARY OF THE INVENTION

The present invention is a method and apparatus for acquiring blood flow information from a subject by illuminating tissues with electromagnetic energy, detecting resulting electromagnetic energy emitted from the tissues and producing an electrical signal proportional thereto; detecting pulsations in the electrical signal at a frequency substantially the frequency of the subject's heart rate; and calculating values from the size of the detected pulsations which are indicative of blood volume pumped by the heart.

A general object of the invention is to provide a non-invasive or minimally invasive method for measuring the hemodynamic performance of the heart. The electrical signal may be produced by detecting light from an illuminated finger or earlobe and the size, or area, of detected pulses in this signal are indicative of the volume of blood pumped by the heart during each heart beat.

Another object of the invention is to provide further information regarding the performance of the subject's heart. The blood volume information may be used in combination with other acquired cardiac function parameters such as ECG or blood pressure to detect compromising cardiac events.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a workstation which employs a preferred embodiment of the invention;

FIG. 2 is an electrical schematic diagram of a data acquisition module which forms part of the workstation of FIG. 1;

FIG. 3 is a graphic illustration of an ECG signal and an electrical signal acquired according to the present invention on the workstation of FIG. 1; and

FIG. 4 is a flow chart of the steps performed by the workstation of FIG. 1 to analyze the electrical signal of FIG. 3.

### DETAILED DESCRIPTION OF THE PREFERRED EMBODIMENT

The present invention may be implemented in a number of different ways. In the preferred embodiment it is implemented in a stand-alone computer workstation; however, it can be appreciated that some or all of the functions may be carried out in other systems.

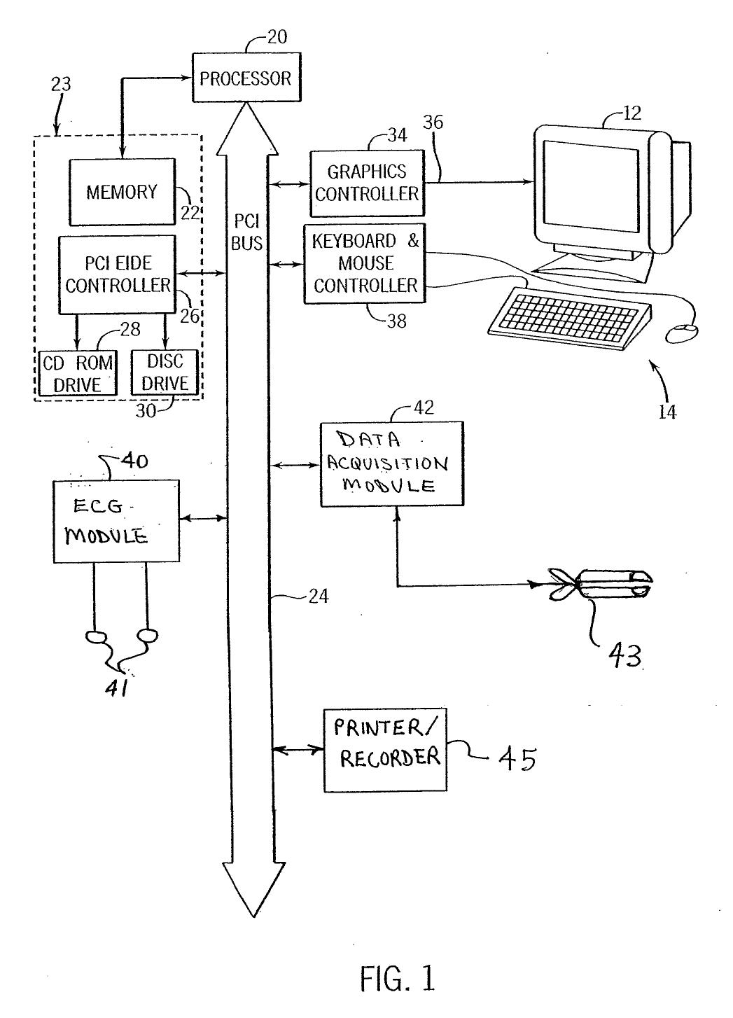

Referring particularly to FIG. 1, the computer workstation includes a processor 20 which executes program instructions stored in a memory 22 that forms part of a storage system 23. The processor 20 is a commercially available device designed to operate with one of the Microsoft Corporation Windows operating systems. It includes internal memory and I/O control to facilitate system integration and integral memory management circuitry for handling all external memory 22. The processor 20 also includes a PCI bus driver which provides a direct interface with a 32-bit PCI bus 24.

The PCI bus 24 is an industry standard bus that transfers 32-bits of data between the processor 20 and a number of peripheral controller cards. These include a PCI EIDE controller 26 which provides a high-speed transfer of data to and from a CD ROM drive 28 and a disc drive 30. A graphics controller 34 couples the PCI bus 24 to a CRT monitor 12 through a standard VGA connection 36, and a keyboard and mouse controller 38 receives data that is manually input through a keyboard and mouse 14.

The PCI bus 24 connects to an ECG module 40 which receives signals from two or more electrodes 41 attached to the subject being examined. It produces a digitized record of the ECG signals for real time display on the CRT 12 and for storage in memory 23.

The PCI bus 24 also connects to a data acquisition module 42. As will be described in more detail below, the module 42 connects to a sensor 43 which fastens to the finger of a subject and produces a signal indicative of light that emanates from tissues in the finger that have been illuminated. This signal is amplified, filtered and digitized by the module 42 so that it can be processed under the direction of a stored program by the processor 20.

The PCI bus also connects to a printer or recorder 45. The recorder 45 is a commercially available device used to print digitized electrical signals in graphic form on a roll of paper. In this system the recorder may print out the ECG signals and simultaneously print out in graphic form the blood flow values produced according to the present invention.

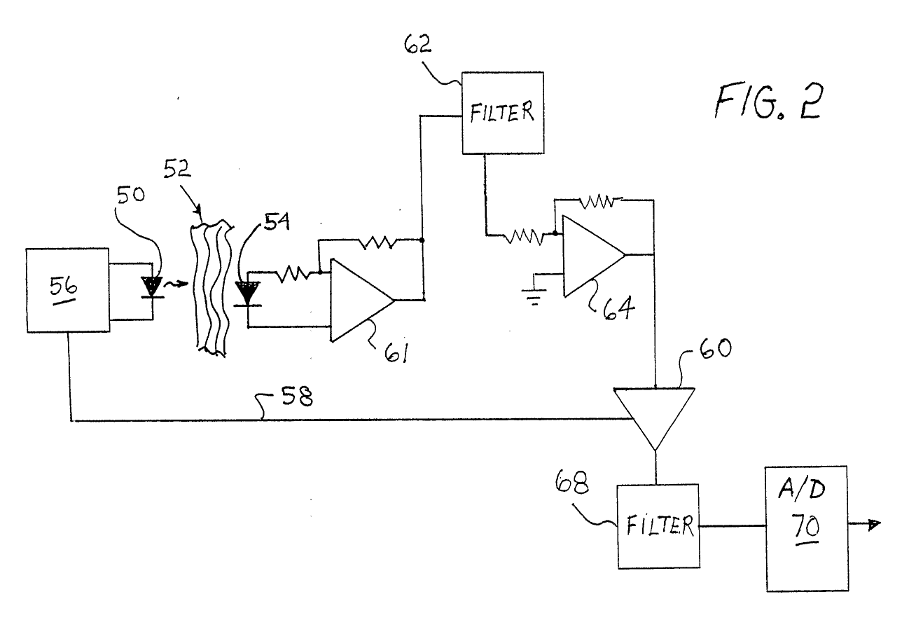

Referring particularly to FIG. 2, the sensor 43 includes a light emitting diode (LED) 50 that produces pulses of infrared light that are directed into a tissue bed 52, and a photodiode 54 that collects and detects light emanating from the tissue bed 52. This detected light may have passed through the tissue 52 (transmitted light) or it may be reflected light. The data acquisition module 42 includes a LED driver circuit 56 which applies current pulses to the LED 50 at a rate of 300 Hz. The LED driver 56 also produces a 300 Hz reference signal on line 58 which is used by a demodulator 60 as will be described below to detect the amplitude modulated signal that results from modulating the light source.

The signal produced by photodiode 54 is amplified by a transimpedance amplifier 61 and applied to the input of a high pass filter 62. The high pass filter 62 is a high pass Butterworth filter having a cutoff frequency of 0.3 Hz. The desired blood flow information is contained in the frequency range of 0.5 Hz to 30 Hz and the high pass filter 62 blocks the DC component of the signal and low frequency noise.

The high pass filtered signal is then amplified by amplifier 64 and applied to one input on the demodulator 60. The demodulator 60 is a four quadrant analog multiplier which demodulates the modulated electrical signal to produce an analog signal that fluctuates in magnitude as a function of the magnitude of the detected light emanating from tissues 52. By modulating the light directed at the tissues 52 and then demodulating the resulting signal using the 300 Hz reference, unmodulated ambient light which might reach the photodetector 54 has no effect on the signal.

The demodulated signal is then applied to a low pass filter 68. The low pass filter 68 has a cutoff frequency of 30 Hz to block high frequency noise. The demodulated and filtered signal is then applied to the input of an analog-to-digital converter 70. The A/D converter 70 samples the analog signal at a rate well above 30 Hz, digitizes the sample, and presents it on the PCI bus 20. These digital samples are continuously read by the processor 20 and stored in memory 23. In most applications these signals will be analyzed in real time, however, in some applications they may be stored and analyzed later.

Many variations are possible in the design of the data acquisition module. When ambient light is not an issue the illumination source need not be modulated and the demodulator 60 may be eliminated. The filtering steps can also be done digitally following conversion of the electrical signal to digital form, in which case the filters 62 and 68 can be eliminated. In other words, the data acquisition module 42 may comprise as little as an amplifier 61, 64 and an A/D converter 70.

While infrared light is used in the preferred embodiment, electromagnetic energy at other frequencies may also be employed.

Referring particularly to FIG. 1, the workstation operates in response to a stored program to analyze the acquired signal and produce blood flow information. The workstation can be configured using this program to display the blood flow information on the CRT display 12, print or record the information using the printer/recorder 45, or store the information for later use in memory 23. In addition, the program may simultaneously input related ECG information from the ECG module 40 and display, print or store the ECG record along with the blood flow record. As indicated above, this analysis and display can be done off-line, in which case the acquired data and related ECG information is stored in memory 23, or it can be done in real time as that information is acquired. In the latter case the analysis program runs in the background on data stored in memory 23 by a timed interrupt program which continuously reads data from the data acquisition module 42 and the ECG module 40.

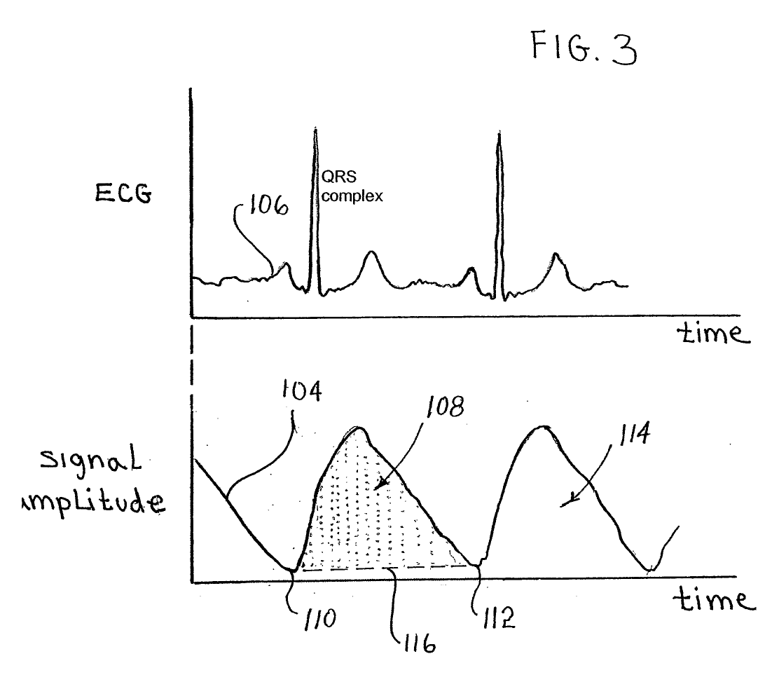

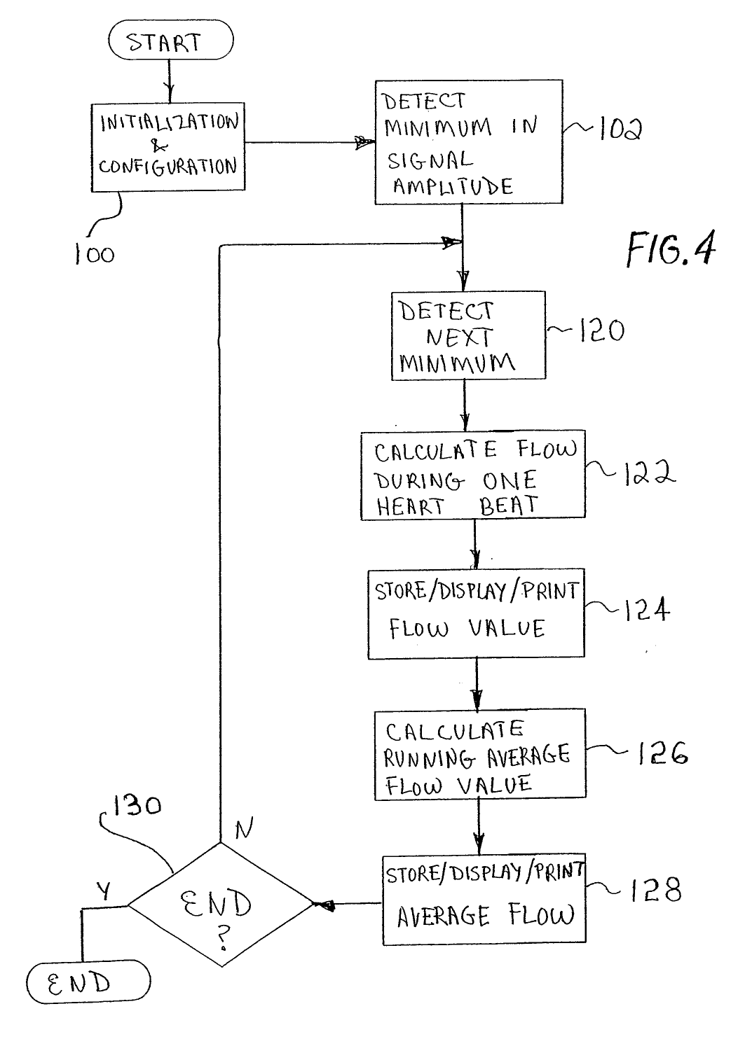

Referring particularly to FIG. 4, after the workstation is configured as described above and indicated at process block 100, the acquired data is examined at process block 102 to detect a minimum in the signal amplitude. As shown in FIG. 3, the acquired electrical signal 104 pulsates in amplitude in synchronism with the subject's heart beat as indicated by the ECG signal 106 acquired at the same time. Each pulsation in the acquired signal 104 is bounded by two signal minimums. For example, the acquired signal pulse 108 is bounded by a first signal minimum 110 and a second signal minimum 112. The second signal minimum also bounds and is the first signal minimum for the next signal pulse 114.

Referring particularly to FIGS. 3 and 4, after the initial signal minimum is detected the acquired data is examined to detect the next signal minimum as indicated at process block 120. If the program is running in real time, this will usually require the system to wait until sufficient signal samples have been acquired and stored in memory 23 as described above. Otherwise, the previously acquired signal data stored in memory 23 is examined to locate the next minimum value.

As indicated at process block 122, once the boundary of a signal pulse has been detected, the area of the signal pulse is calculated. The area of the signal pulse has been found to be directly related to the quantity of blood flowing through the illuminated tissue, and hence directly related to the total blood flow pumped by the heart during that heart beat. There are numerous ways in which the area of a signal pulse can be calculated, but in the preferred embodiment the area beneath one signal pulse 108 is calculated by integrating the acquired signal samples between the two minimums 110 and 112 and then subtracting the area beneath the line indicated at 116 which connects the two minimums 110 and 112. This calculated area is the measured blood flow for one heart beat.

As indicated by process block 124, the calculated flow value may be stored, displayed or used to print a record, depending on how the system is configured. This may be a numeric value, or a point on a graph. Because signal artifacts can sometimes corrupt the measurement, it has been found useful to also calculate a running average of the calculated flow values as indicated by process block 126. In the preferred embodiment the output of this digital filter is the average of the five most recently calculated flow values. As indicated at process block 128, these filtered flow values are also displayed, stored or printed as determined during system configuration.

The system may also analyze the calculated blood flow values to derive further information of clinical importance. It can be appreciated that the blood flow values are not calibrated to measure an actual blood volume, but are directly related to the actual blood volume pumped by the subject's heart. One clinical value of this blood flow information resides in the changes that occur, rather than the absolute values. Thus, when a compromising cardiac event occurs the heart will pump less blood and this will be detected as a drop in the blood flow values. Such changes can be seen in a graphic display of the blood flow values, or values which indicate the change in blood flow values can be calculated. Thresholds can be established and when the change in blood flow exceeds such a threshold, a programmed event can be signaled.

As indicated at decision block 130, the system then loops back to analyze the next acquired pulse in the same manner until all the stored data has been analyzed or the operator terminates the process.

While the invention has been described in the context of a workstation, many other embodiments are possible. For example, the functions of the data acquisition module 42 and ECG module 40 may be embodied in a portable Holter. In this clinical application the sampled signals are recorded for a time period, and if a cardiac event is detected, those recorded signals are saved for later analysis at a workstation. In this case the blood flow data helps the diagnostician determine if the recorded cardiac event detected by ECG signals had a hemodynamic impact on the patient.

In a bed side monitor embodiment of the present invention most of the hardware depicted in FIG. 1 is housed in an instrument that can be positioned near the subject's bed. In addition to the data which is recorded and or displayed, the analysis software in this instance will also produce an alarm that is signaled when a cardiac event of concern is detected. In this clinical application blood flow data is employed in the analysis along with other cardiac parameters such as blood pressure and ECG to determine if a compromising hemodynamic event has occurred.

1. A method for indicating blood flow produced by a subject's heart, the steps comprising:
a) illuminating with electromagnetic energy tissue on the subject which is perfused with blood;
b) detecting electromagnetic energy emanating from the tissue to produce an electrical signal;
c) detecting signal pulses in the electrical signal which correspond to beats of the subject's heart; and
d) producing a series of flow values by calculating the areas of successive signal pulses.

2. The method as recited in claim 1 which includes:
filtering the electrical signal to pass frequencies in the range of 0.5 Hz to 30 Hz.

3. The method as recited in claim 1 which includes:
modulating the electromagnetic energy illuminating the tissue at a selected frequency; and
step b) includes detecting the electromagnetic energy with a photodetector and demodulating a signal produced by the photodetector.

4. The method as recited in claim 1 in which step b) includes:
producing an analog signal proportional to the electromagnetic energy emanating from the tissue; and
converting the analog signal to a digital signal.

5. The method as recited in claim 1 in which step c) includes:
detecting minimum amplitudes in the electrical signal which define the boundaries of the signal pulses.

6. The method as recited in claim 5 in which step d) includes calculating the area beneath the electrical signal and between successive minimum amplitudes.

7. The method as recited in claim 1 which includes filtering the series of flow values by calculating a running average of a selected plurality of said flow values.

8. The method as recited in claim 1 which includes acquiring a signal indicative of a measured cardiac function; and
displaying the series of flow values simultaneously with the display of the acquired cardiac function signal.

9. The method as recited in claim 8 in which the acquired cardiac function signal is an ECG signal.

10. The method as recited in claim 9 which includes:
determining the occurrence of a significant cardiac event by using the ECG signal and the flow values.

11. A system for evaluating cardiac function of a subject which includes:
a sensor for detecting energy emanating from perfused tissue in the subject and producing an electrical signal proportional thereto;
a pulse detector connected to receive the electrical signal and detect signal pulses therein which correspond to beats of the subject's heart; and
a calculator connected to the pulse detector and being operable to produce a blood flow indication by calculating the size of the detected signal pulses.

12. The system as recited in claim 11 in which the calculator computes a flow value corresponding to the area of a detected signal pulse.

13. The system as recited in claim 11 in which the sensor includes a light emitting device which illuminates the perfused tissue and a light detector which produces an electrical signal proportional to light emanating from the perfused tissue.

14. The system as recited in claim 11 in which the signal detector includes a filter which suppresses frequencies in the electrical signal outside a range of frequencies which includes the frequency of the subject's heart beat.

15. The system as recited in claim 14 in which said range of frequencies is substantially 0.5 Hz to 30 Hz.

16. The system as recited in claim 1 in which the pulse detector includes means for detecting minimum values in the electrical signal which define the boundaries between successive signal pulses.

17. The system as recited in claim 16 in which the calculator computes the area beneath the electrical signal amplitude between successive detected minimums.

18. The system as recited in claim 11 in which the calculator computes a running average of the sizes of a predetermined number of signal pulses.

19. The system as recited in claim 11 which includes an analyzer which receives the blood flow indication produced by the calculator and produces therefrom a signal indicative of cardiac function.

20. The system as recited in claim 19 in which the analyzer calculates changes which occur in the blood flow indication.

21. The system as recited in claim 19 which includes:
means for acquiring a signal indicative of a cardiac parameter which measures a selected cardiac function; and
the analyzer produces from the blood flow indicator and the signal indicative of a cardiac parameter a signal indicative of cardiac event.

22. The system as recited in claim 21 in which the means for acquiring a signal indicative of a cardiac parameter is an ECG module.

23. The system as recited in claim 22 which includes memory for storing the electrical signal and the signal indicative of a cardiac parameter produced over a period of time.
